(19) United States
(12) Patent Application Publication
(10) Pub. No.: US 2011/0184154 A1
(43) Pub. Date: Jul. 28, 2011

(54) CELL BROTH CLARIFICATION AND HOST CELL PROTEIN REMOVAL

(75) Inventors: Grigorios Zarbis-Papastoitsis, Watertown, MA (US); Michael Christopher Kuczewski, Newton, MA (US); Emily Belcher Schirmer, Everett, MA (US)

(73) Assignee: PERCIVIA LLC, Cambridge, MA (US)

(21) Appl. No.: 13/083,538

(22) Filed: Apr. 9, 2011

Related U.S. Application Data
(63) Continuation-in-part of application No. PCT/EP2009/063568, filed on Oct. 16, 2009.
Application No. PCT/EP2009/063568 is a continuation-in-part of application No. PCT/EP2009/063566, filed on Oct. 16, 2009.
Application No. PCT/EP2009/063566 is a continuation-in-part of application No. PCT/EP2009/063565, filed on Oct. 16, 2009.
(60) Provisional application No. 61/106,328, filed on Oct. 17, 2008.
(60) Provisional application No. 61/106,324, filed on Oct. 17, 2008.
(60) Provisional application No. 61/106,320, filed on Oct. 17, 2008.

(30) Foreign Application Priority Data
Oct. 24, 2008 (EP) EP20080167506
Oct. 24, 2008 (EP) EP20080167508
Oct. 24, 2008 (EP) EP20080167509

(51) Int. Cl. C07K 1/14; C12N 5/071; C12N 1/02

(52) U.S. Cl. 530/389.1; 435/325; 435/261; 435/317.1

(57) ABSTRACT

The present invention relates to a method for clarification of, and removal of host cell proteins from, a cell broth consisting essentially of viable cells, a culture medium and a secreted desired biological substance having an overall positive charge in the cell broth by contacting the cell broth with a particulate anion exchanger, allowing an adequate incubation time to result in formation of a cell pellet and a supernatant layer, separating the resulting cell pellet from the supernatant layer. The present invention further relates to a method for the recovery of a secreted desired biological substance from the cell broth by extracting the secreted desired biological substance from the supernatant layer.

### FIELD OF THE INVENTION

The present invention relates to a method for the clarification of a cell broth containing cells secreting desired biological substances, to facilitate the reduction of undesired host cell protein (HCP) and DNA, and the high yield recovery of highly purified secreted desired biological substances therefrom.

### BACKGROUND OF THE INVENTION

In the past few years biotechnology manufacturing has demonstrated major improvements in monoclonal antibody (MAb) production with product titers as high as 25 g/L, which are often associated with very high cell densities (Golden et al. 2009). The eXtreme Density (XD®) cell culture process is a continuous process where both cells and product are retained in a stirred tank bioreactor using suspension culture of PER.C6® human cells (Golden et al. 2009; Zijlstra et al. 2008). This is accomplished by the use of a modified alternating tangential flow perfusion system where fresh medium is continuously supplied and waste by-products are continuously removed and discarded. In this way, cell densities of over 150 million viable cells/mL and product titers of over 25 g-MAb/L are possible. Because the product is retained inside the bioreactor, the XD® process produces a much lower volume of harvest (only that which is contained in the bioreactor), requiring much less downstream processing than traditional perfusion processes (Golden et al. 2009).

High density cell cultures with upwards of 150 million cells/mL pose a great challenge in clarification and further downstream processing due to the need to remove a large amount of biomass and the increased levels of contaminants including cell debris that is generated during the cell culture process. The production of biological substances, in particular MAbs, usually involves processing a complex cell culture broth from which the desired biological substances must be isolated and purified while maintaining high overall product recovery and quality. In many instances the biological substances are present extracellularly and will thus be present in the cell broth fluid. As a first step, solid material such as the cells and cell debris are separated from the cell broth—a step called clarification.

Clarification methods used in the prior art include centrifugation, filtration (such as microfiltration, depth filtration and filtration through absolute pore size membranes) and expanded bed chromatography. Traditionally, centrifugation and a combination of filtration techniques (tangential flow filtration and depth filtration) have been widely accepted as the workhorses for clarification of these complex cell culture broths (Lutz et al. 2009; Pham 2007; Shukla and Kandula 2009). However, with the development of improved and more efficient mammalian cell culture processes where the total cell density can reach far beyond traditional levels of 20×10⁶ cells/mL for CHO cells, (Jayapal et al. 2007) to over 150×10⁶ cells/mL for PER.C6® cells (Golden et al. 2009; Zijlstra et al. 2008) the limitations of both centrifugation and filtration techniques are quite apparent due to the high solids (up to 40%) content of these harvests.

While centrifugation can be applied to process feed streams with high levels of solids, the product recovery could be low due to the increased pellet volume and need to frequently de-sludge (especially in large-scale continuous centrifugation). Additionally, cell disruption from shear forces generated during centrifugation can further decrease the efficiency of harvest clarification (Pham 2007) and potentially result in product damage (Schmidt 2009) and/or entrapment.

Depth filters are advantageous because of their ability to remove contaminants (Yigzaw et al. 2006), and many depth filters are available in a single-use format reducing the need for cleaning and validation (Pailhes et al. 2004). However, depth filters are currently not able to handle feed streams with high solids content and are often used in series with centrifugation.

Tangential flow filtration (TFF) is advantageous because of its ability to handle high solids loading, but this technique can exhibit poor yield due to polarization of solids at the membrane surface when processing highly dense feed streams. Moreover, excessive product dilution and cell lysis due to shear forces can also limit the utility of TFF.

### Reported Developments

Flocculation of cell culture harvests has been widely used to enhance clarification throughput and downstream filtration operations (Akeprathumachai et al. 2004; Kim et al. 2001; Riske et al. 2007). These prior art flocculation methods may be employed in order to enhance any of these clarification methods, in particular in combination with filtration. Such methods typically use soluble synthetic polyionic polymers (such as DEAE dextran, acryl-based polymers, polyethylene amine), naturally derived polymers (chitosan) or inorganic materials such as diatomaceous earth or perlites (Suh et al. 1997).

Disadvantages of the use of the prior art flocculation agents are, amongst others, that they may bind the desired biological substances of interest, that they may inactivate the desired biological substances of interest, that the flocculation process takes too long and/or that the flocculation agent may be hard or expensive to prepare in the high quality needed for medical use. The soluble polymers used in these methods must be removed from process streams and their removal requires monitoring and quantification by in-process and product release assays. Furthermore, if ion exchange (IEX) chromatography is included as a purification step in the downstream process, the IEX binding capacity will be greatly affected due to the charged nature of the soluble flocculant. Additionally, the high viscosity of polycation stock solutions presents a further process challenge.

US patent application 2003/170810 discloses a method wherein a crude cell lysate is clarified by mixing the lysate with an anion exchange resin in a batch and then separating the insoluble material (including the resin and anything bound thereto, cells and cell debris) from the soluble material via filtration, centrifugation or gravity separation. The cell lysis step introduces a variety of cellular impurities and debris requiring additional purification steps not required by the present invention.

WO2007/108955 discloses a protein purification process starting from the pre-clarified supernatant of a cell culture, in particular the purification of antibodies or antibody-like proteins, using cation exchange followed by anion exchange chromatography, and may be carried out using a batch process followed by a separation step (separation of supernatant from solid material). WO2007/108955 does not disclose the use of the chromatography steps on an unclarified cell broth.

WO2008/079280 discloses the purification of biomolecules from a mixture, by adding soluble polymers (e.g. soluble ion exchange polymers) to the mixture and changing the conditions so as to precipitate these and the biomolecules bound to it, thus separating the biomolecules into fractions. WO2008/079280 does not disclose the use an insoluble particulate ion-exchange material or suggest a purification process carried out on a cell broth.

The present invention permits the reduction in the number of protein purification steps from a complex cell broth mixture. Furthermore, the prior art clarification methods have been shown to be effective only at relatively low cell densities, and with cell lysates. Also, most of these methods were not shown to be successfully applied to mammalian cells, in particular not to mammalian cells which produce secreted desired biological substances.

An object of the present invention is to provide an alternative to centrifugation and prior art flocculation techniques to facilitate commonly used micro- or depth-filtration steps for the clarification of cell harvests.

A further object of the present invention is to provide for a method of cell broth clarification wherein the cell broth consists essentially of viable cells with minimal cell lysate, which further facilitates the recovery and purification of the desired secreted biological substances in high yield.

### SUMMARY OF THE INVENTION

The present invention relates to a method for the clarification of a cell broth comprising the steps of:
(a) forming a mixture by contacting a particulate anion exchange material with a cell broth consisting essentially of culture medium, a desired biological substance having an overall positive charge in said cell broth, host cells that have produced said desired biological substance and being substantially viable, and host cell proteins,
(b) incubating said mixture for an adequate time to result in the formation of a cell pellet containing substantially all of said host cells and said particulate anion exchange material, and a supernatant layer containing said desired biological substance, and
(c) separating the resulting cell pellet from said supernatant layer.

The present invention is advantageous over prior art clarification techniques because the present anion exchange material is an insoluble particulate material, preferably an anionic polymer attached to an insoluble matrix (such as ion exchange chromatography matrices), which insoluble particulate material is removed with the host cells from the cell broth. Accordingly, the particulate anion exchange materials used in the present invention induce and enhance the settling of cells in situ, forming a partially clarified supernatant, with a much lower cell density than the starting material, which clarified supernatant facilitates further cost-effective processing, for example, by depth filtration.

Since the present invention uses matrices having ionogenic groups, the present method also facilitates the reduction of contaminants such as HCP and DNA. A reduction of these impurities at this early stage of the downstream process greatly increases the efficiency of subsequent unit operations, such as affinity or ion exchange chromatography, and thus reduces the overall number of steps required for downstream processing.

Another embodiment of the present invention relates to a method for the recovery of a desired biological substance from a mammalian cell broth containing host cells secreting said desired biological substance, comprising the steps of:
(a) forming a mixture by contacting a particulate anion exchange material with a cell broth containing cell culture medium, host cell proteins, substantially viable mammalian host cells, and a desired biological substance secreted by said mammalian host cells, said substance having an overall positive charge in said cell broth, wherein said mammalian host cells are present at an initial cell density of at least about 15×10⁶ cell/ml,
(b) incubating said mixture for an adequate time to result in the formation of a cell pellet and a supernatant layer containing the desired biological substance and having a reduced cell density and host cell protein content,
(c) separating the resulting cell pellet from the supernatant layer, and
(d) extracting the desired biological substances from the supernatant layer.

A particularly preferred embodiment uses a particulate anion exchange material having a specific density of the particles of between about 1.4 and about 3 g/ml.

### BRIEF DESCRIPTION OF THE DRAWINGS

FIG. 1. Recovery of product after initial settling with Si-PEI and subsequent washing steps with PBS.

FIG. 2. Supernatant cell density as a function of time for various resins. The cell densities were measured by Vi-CELL (Beckman-Coulter).

FIG. 3. Supernatant volume as a function of time for various resins. The total volume in each case was 24 ml.

FIG. 4. Shows the SDS-PAGE and agarose gels of samples taken from each of the reactors as described in Example 6. A) SDS-PAGE of reactor 1 and 2 samples B) DNA-Agarose gel of reactor 1 and 2 samples. Samples were pre-treated with protein A to remove the MAb. Lanes: 1-MW standard or 1 kBase ladder; 2-Reactor 1 harvest; 3-Reactor 1 Si-PEI treated partially clarified harvest; 4-Reactor 2 harvest; 5-Reactor 2 Si-PEI treated partially clarified harvest.

### DETAILED DESCRIPTION

The following definitions are used in the following description to assist in understanding the scope of the present invention.

The term “anion exchange material” means weak or strong anion exchange chromatography media.

The term “biological substance” means a chemical substance produced by a biological entity. Exemplary biological substances produced by the host cells (for example by expressing a (recombinant) gene coding therefore) are, for example, organic compounds, and complex systems and macromolecules, such as viruses or (recombinant) proteins, in particular receptors, enzymes, fusion proteins, blood proteins such as proteins from the blood coagulation cascade, multifunctional proteins such as for instance erythropoietin, virus or bacterial proteins for instance for use in vaccines; immunoglobulins such as antibodies, for example IgG or IgM, and the like. Preferred biological substances are preferably polypeptides, or proteins. The most preferred biological substances are immunoglobulins or portions or fragments thereof. In the context of the present invention, the terms ‘product’ and ‘biological substance’ are interchangeable. Preferably, the biological substances such as proteins or vaccines can be used as an active ingredient in a pharmaceutical preparation.

The term “cell” means prokaryotic cells, eukaryotic cells, phage particles, and organelles.

The term “cell broth” means a cell culture inoculated with viable cells. Preferred examples of cell broths also contain culture medium, as well as secreted biological substances.

The term “cell density” means the concentration of cells in a solution, culture medium or supernatant layer (e.g., cells/mL). The symbol “Xₜ” means total cell density in units of cells/mL. The symbol “Xv” means the cell density of viable cells per mL. The cell density, and the relative amount of live versus dead cells, can be measured using a cell counter such as Vi-CELL™ (with the trypan blue exclusion method). Cell density may also be measured by cytometry, packed cell volume determination, or Coulter counters (with the Electrical Sensing Zone Method).

The term “contacting” means the physical mixture of two or more materials. A preferred “contacting” according to the present invention mixes anion exchange material with the cells contained in a cell broth.

The term “culture medium” means the extracellular environment containing the nutrients and other constituents supporting the growth and production of cells, but may also contain waste products or host cell proteins (HCP) or material from lysed cells. The composition of the culture medium may vary in time during the course of the culturing of cells and at the stage of clarification may be depleted of one or more of the original constituents.

The term “desired” means the biological substance that is produced by the cells in the cell broth and which is intended to be isolated and purified.

The term “host cells” means cells that produce, or that have been bioengineered to produce, and secrete extracellularly a biological substance. Preferred host cells are mammalian cells, examples of which include CHO (Chinese Hamster Ovary) cells, hybridomas, BHK (Baby Hamster Kidney) cells, myeloma cells, human cells, for example HEK-293 cells, human lymphoblastoid cells, E1 immortalized HER cells, mouse cells, for example NS0 cells. More preferably, E1 immortalized HER cells are used, most preferably PER.C6® cells. In a preferred embodiment, the cells in the process of the present invention are E1-immortalized HER cells, more preferably PER.C6® cells (see U.S. Pat. No. 5,994,128, the content of which is incorporated by reference here). PER.C6® cells are deposited under ECACC No. 96022940 (see, e.g., U.S. Pat. No. 5,994,128, EP 0833934 B1, the contents of which are incorporated by reference here).

The term “host cell proteins” or “HCP” means extraneous proteins produced by the host cells in the cell broth and that do not comprise the desired biological substance intended to be produced, isolated and purified.

The term “incubating for an adequate time” means the time in which the precipitation of the cells results in a distinct cell pellet volume and a supernatant layer. Preferred adequate times range from about 30 minutes to three hours, more preferably from 45 minutes to 2.5 hours, and most preferably from about one hour to about 2 hours.

The terms “lysate” or “cell lysate” refer to a composition consisting essentially of cells that have ruptured cell walls and/or cell membranes. “Crude lysate” refers to a lysate that has not been fractionated to remove one or more cellular components. “Clarified lysate” refers to a cell lysate that has been fractionated to remove one or more cellular components, such as cell debris and other insoluble materials, cell wall and/or cell membrane materials, lipids, insoluble proteins, nucleic acids, including DNA and RNA.

The term “lysing,” with reference to a cell suspension, refers to rupturing the cell walls and/or cell membranes of at least a portion of the cells such that at least part of the contents of the cells are released.

The term “overall positive charge” means that the electrostatic contribution of positive and negatively charged ionogenic groups on a substance in its fluid environment results in a net positive charge. The preferred overall positive charge is determined with respect to the secreted desired biological substances, wherein the overall charge is based on the pKₐ of the acidic and basic residues comprising the substance and the pH of the aqueous environment—in this case, the pH of the cell broth. For the biological substance to have a net positive charge in the cell broth, the pI (the pH where the net charge is zero) of the substance must be higher than the pH of the cell broth.

The term “PBS” means phosphate buffered saline.

The term “recovery” means obtaining the desired product essentially free from host cell proteins and host cell DNA, and other contaminants.

The term “secreted biological substances” means biological substances that the cells produce, and release (i.e., secrete) extracellularly.

The term “separating” means any method to remove the supernatant from the cell pellet, such as by decanting or drawing out the supernatant or e.g. by draining the pellet from the vessel through a port at the bottom.

The term “supernatant layer” means the liquid overlying volume as a result of the settling. The supernatant layer may (and generally will) still contain cells, be it at a cell density significantly lower than the initial cell density.

The term “viable” as it relates to a cell means a live cell.

The term “% viability” means the percentage of live host cells in a cell broth.

The following symbols and further abbreviations are used throughout the specification:
CM Carboxymethyl
DEAE Diethylaminoethyl
DNA Deoxyribonucleic acid
ECS Enhanced cell settling
ELISA Enzyme-linked immunosorbent assay
g Gravitational force
IEX Ion Exchange
MAb Monoclonal Antibody
n.d. Not determined
PEI Polyethyleninimine
pI Isoelectric point
PrA HPLC Analytical Protein A High Pressure Liquid Chromatography
rt-PCR Real time polymerase chain reaction
SDS-PAGE Sodium Dodecylsulfate Polyacrylamide Gel Electrophoresis
Si-PEI Bakerbond Wide-Pore PEI
TBE Tris borate EDTA (ethylenediaminetetraacetic acid)
TFF Tangential flow filtration
TP Toyopearl—class of IEX materials from Tosoh Biosciences.
Vₚₒₒₗ Volume of pooled supernatant and washes, cm³
V_{work} Working volume of bioreactor, cm³
XD® eXtreme Density (Perfusion Bioreactor)

### DESCRIPTION OF THE EMBODIMENTS

The anion exchange material used in the present invention generally comprises a carrier, which may be organic material or inorganic material or a mixture of organic and inorganic material. Suitable organic materials are agarose based media and methacrylate. Suitable inorganic materials are silica, ceramics and metals. The particles preferably may have a size of between about 15 and about 150 m. More preferably their size is between about 15 and about 70 m. The particles should have a density suitable for effecting relatively rapid sedimentation of the cells from the cell broth, but not above the density that the present inventors have determined does not operate to effect the sedimentation. In a specific embodiment of the present invention, the specific density of the particulate anion exchange material is between about 1.4 and about 3 g/ml. Most preferably, the particle density is about 2 g/ml. A method suitable for determination of the particle density of the anion exchange material is described in the Examples section. Suitable anion exchange materials that fulfill this requirement are materials with particles made of or containing silica, ceramic material or a metal core.

The cell broth for clarification may be obtained by any cell culturing method suitable for attaining a cell density of at least 15×10⁶ cells/ml. Particularly suitable methods in this respect are described in e.g. WO2005095578, WO2004099396 and WO2008006494, the contents of which are incorporated herein by reference. These methods may yield mammalian cells at very high cell densities. The preferred cell broths consist essentially of viable host cells. Specifically, the cell broth should contain no more than about 22% of said host cells as dead cells, preferably no more than about 10% of said host cells as dead cells, more preferably no more than about 5% of said host cells as dead cells, and most preferably no more than about 3% of said host cells as dead cells.

A preferred method of the invention introduces the particulate anion exchange material into the cell broth when the total cell density is no less than about 90% of the cell density maximum (“Xt-max”), and, more preferably, when the total cell density is not less than 95% of Xt-max, and most preferably, when the total cell density is no less than about 1% of Xt-max.

As a practical upper limit, the process according to the present invention may be carried out with cell densities up to about 200×10⁶ cells/ml, preferably about 175×10⁶ cells/ml, and more preferably up to about 130×10⁶ cells/ml. The method according to the present invention is particularly useful for cell broths containing mammalian cells at very high cell density, such as about 60×10⁶ cells/ml, but even for cell densities as high as about 120×10⁶ cells/mL.

In the process of the present invention, particularly when the cell density is extremely high, it may also be desirable to dilute the starting material from the bioreactor to a preferred cell density. If the initial cell density is above 130×10⁶ cells/ml it is advisable to first dilute the cell broth. In practice, it is preferred that a cell broth with an initial cell density above 100×10⁶ cells/ml be first diluted. Dilution preferably may be done to a cell density of at least about 15×10⁶ cells/ml but not more than about 80×10⁶ cells/ml. The cell broth may be diluted with a solution that does not greatly change the environment of the cell so as to not cause lysis of the mammalian cells, i.e. an isotonic solution such as PBS.

The method of the present invention results in the formation of a supernatant layer containing the desired biological substance and wherein the density of host cells remaining therein said supernatant layer is reduced by at least about 87 percent, preferably about 90 percent, and most preferably about 98 percent, to from about 2×10⁶ cell/ml to about 15×10⁶ cell/ml. Furthermore, the present inventors surprisingly found that the method according to the present invention also results in a considerable reduction of the host cell protein content in the clarified supernatant layer.

The present invention further relates to a method for the recovery of secreted desired biological substances from a cell broth containing cells producing the secreted desired biological substance having an overall positive charge as described above and wherein the cells in the cell broth are mammalian cells at an initial cell density of at least 15×10⁶ cells/ml, wherein the resulting cell pellet is further processed by the following steps:
(e) re-suspending said resulting cell pellet from step (c) after separation of said supernatant layer in an aqueous salt solution to form a second mixture,
(f) incubating said second mixture for an adequate time to allow the formation of a second cell pellet and a second supernatant layer from said re-suspended cell pellet,
(g) separating said second cell pellet from said second supernatant layer, and
(h) extracting the desired biological substances from said second supernatant layer.

In a further aspect according to the present invention, step (f) through (h) of the above process are repeated one or more times.

In a further preferred method according to the present invention the resulting cell pellet is re-suspended in an aqueous solution that does not alter the environment of the cell to avoid lysis, such as an aqueous (preferably isotonic) salt solution, more preferably, PBS.

Preferably the first, second and subsequent supernatant layers are collected and the secreted desired biological substance is extracted from the pooled supernatants.

Suitable methods for extracting the secreted desired biological substances from the supernatant layer are for example filtration (such as depth filtration, microfiltration, ultrafiltration, diafiltration), chromatography (such as size exclusion chromatography, affinity chromatography, cation exchange chromatography, hydrophobic interaction chromatography, immobilized metal affinity chromatography), aqueous two-phase extraction, precipitation or centrifugation. Advantageously, the desired biological substance can be extracted very efficiently by cation exchange chromatography. In case of immunoglobulins as the desired biological substances affinity chromatography, in particular protein A chromatography, and cation exchange chromatography are especially suitable separation methods.

A special embodiment of the extraction of secreted desired biological substances is wherein said combined supernatant layer is subjected to depth filtration to further reduce host cell density and host cell protein content.

### EXAMPLES

The following commercially available materials are used in the examples. Additional data on commercial materials described herein are described in Table 1 below:
Si-PEI=Bakerbond Wide-Pore PEI (PolyEthyleneImine) Prep LC Packing grafted silica beads (JT Baker).
DEAE Hyper D=diethylaminoethyl grafted ceramic beads (Pall).
Super Q=quaternary amino functionality on a methacrylate support (Tosoh).
Streamline DEAE=diethylaminoethyl grafted agarose beads (GE Healthcare)
TP DEAE=diethylaminoethyl functionality on a methacrylate support (Tosoh).

The following clarification experiments were performed at various cell densities with PER.C6®® cells bioengineered to produce antibodies and prepared according to the procedure outlined in WO2008006494. The concentration of antibody in the supernatant layers was measured by PrA HPLC. The concentrations were corrected for biomass when necessary, i.e. when the cell density is extremely high, the cells contribute significantly to the working volume.

### Example 1

### Clarification with Low Cell Density

Different amounts of Si-PEI were added to individual vials containing 10 ml of cell culture. Xₜ=4.3×10⁶ cells/ml. The cells were allowed to settle for 15 minutes. Only 5% (vol) of Si-PEI was needed to settle 97% of the cells. Adding 10% (vol) of Si-PEI settled 99% of the cells. Product recovery was 100%.

The addition of Si-PEI greatly reduced the time needed for the cells to settle. Also, the addition of Si-PEI resulted in a more compact pellet in comparison with the control (no Si-PEI added).

### Example 2

### Clarification with Intermediate Cell Density

Different amounts of Si-PEI were added to individual vials containing 5 ml of cell culture. Xₜ=63.5×10⁶ cells/ml. The cells were allowed to settle for 30 minutes. Adding 5% (vol) of Si-PEI settled 87% of the cells. Adding 10% (vol) of Si-PEI settled 89% of the cells. Adding 20% (vol) of Si-PEI settled 85% of the cells. In each case the resulting cell density was below 10×10⁶ cells/ml, which is a suitable feed for depth filtration. Product recovery was 97%.

The addition of Si-PEI greatly reduced the time needed for the cells to settle. Also, the addition of Si-PEI resulted in a more compact pellet in comparison with the control (no Si-PEI added).

### Example 3

### Clarification with High Cell Density

10% (vol) of Si-PEI was added to 345 ml of cell culture broth. Xₜ=123×10⁶ cells/ml. Due to the high cell density two hours of settling were allowed. After these two hours the cell density in the resulting supernatant was 13.6×10⁶ cells/ml. The pellet volume was 53% of the total volume (93% for the control where no Si-PEI was added). The supernatant was decanted and the pellet was washed twice with isotonic PBS with 1 hour of settling after each wash. Product recovery was 93% after the two washes. The total process time was 4 hours. After pooling the supernatants, the final process volume was 600 ml and the cell density was 9.9×10⁶ cells/ml.

### Example 4

### Maximizing Recovery of Product by Repeated Washings

10% (vol) of Si-PEI was added to 345 ml of cell culture broth. Xₜ=78×10⁶ cells/ml (reactor 1) or 120×10⁶ cells/ml (reactor 2). About 200 ml of the supernatant layer were decanted after initial settling. This volume was replaced by an equal volume of PBS, and the cells were allowed to settle for 60 minutes. Again about 200 ml of the supernatant layer was decanted and replaced by an equal volume of PBS followed by settling for 60 minutes. And again 200 ml of the supernatant layer was decanted.

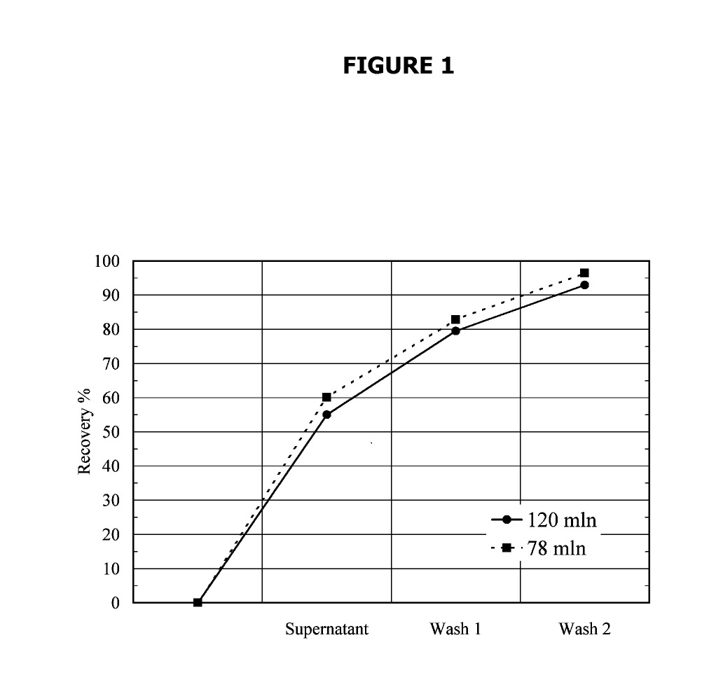

The product recovery through the several washing steps is summarized in FIG. 1. The results show a very considerable improvement of the product recovery by collecting the product from the supernatants after only two washing steps.

### Example 5

### Clarification with Anion Exchange Material of Various Densities

### (A) Method for Determination of Particle Density of Ion Exchange Materials.

The particle density (g dry/ml) of various ion exchange materials was determined pycnometrically. The volume of a 10 mL pycnometer (#15123R-10 Kimble Glass, Inc., Vineland, N.J.) was determined in triplicate as follows:
1. Weigh clean, dry, empty pycnometer (Wᵢ) assembly.
2. Completely fill pycnometer with water at room temperature.
3. Insert thermometer and wipe off excess water at overflow tube. Cap overflow tube.
4. Re-weigh pycnometer (W_{f}) assembly.
5. Volume of pycnometer is determined by:

$${V}_{pyc} = \frac{( {W}_{f} - {W}_{i} )}{{ρ}_{{H}_{2} O} ( T )}$$

where _{H20}(T) is the density of water as a function of temperature.

The particle density was then determined as follows:
a. Weigh clean, dry, empty pycnometer (Wᵢ) assembly.
b. Weigh dry (dried at 50° C. for 3 h) anion exchange material in pycnometer (Wᵣ).
c. Completely fill pycnometer with water at room temperature
d. Insert thermometer and wipe off excess water at over flow tube. Cap overflow tube.
e. Re-weigh pycnometer (W_{f}) assembly.
f. Particle density (_{d}) is determined by:

$${ρ}_{d} = \frac{( {W}_{r} - {W}_{i} )}{[ {V}_{pyc} - \frac{( {W}_{f}^{′} - {W}_{r} )}{{ρ}_{{H}_{2} O} ( T )} ]}$$

The results for a variety of ion exchange materials are summarized in Table 1:

TABLE 1

| | | | Mean | |
|---|---|---|---|---|
| | Manu- | | particle | Particle |
| Ion exchange | facturer/ | Chemistry/ | size | density (g |
| material | Catalog No. | Backbone | (m) | dry/mL) |
| HyperZ | Pall/21012 | 4° amine/ | 75 | 4.08 ± 0.07 |
| | | Zirconium oxide | | |
| Bakerbond WP | JT Baker/ | 1°, 2°, 3° amine/ | 40 | 2.02 ± 0.03 |
| PEI | 7368 | Silica | | |
| Bakerbond WP | JT Baker/ | 1°, 2°, 3° amine/ | 15 | 2.06 ± 0.1 |
| PEI | 7180 | Silica | | |
| CM HyperD | Pall/20050 | Carboxymethyl/ | 50 | 1.92 ± 0.1 |
| | | ceramic | | |
| DEAE HyperD | Pall/ | 3° amine/ | 50 | 1.44 ± 0.08 |
| | 20067 | Ceramic | | |
| Toyopearl | Tosoh/ | 4° amine/ | 65 | 1.22 ± 0.02 |
| SuperQ-650M | 43205 | Methacrylate | | |
| Toyopearl | Tosoh/ | 3° amine/ | 65 | 1.17 ± 0.04 |
| DEAE-650M | 43201 | Methacrylate | | |
| Toyopearl | Tosoh/ | 4° amine/ | 35 | 1.17 ± 0.01 |
| SuperQ-650S | 19823 | Methacrylate | | |
| Toyopearl | Tosoh/ | 3° amine/ | 35 | 1.25 ± 0.05 |
| DEAE-650S | 19804 | Methacrylate | | |

(B) Clarification experiments. Si-PEI (15 and 40 m), ToyoPearl Super Q (35 and 65 m), ToyoPearl DEAE (35 and 65 m), DEAE Hyper D, CM Hyper D and HyperZ were evaluated with a cell broth containing PER.C6®® cells producing a monoclonal antibody (MAb). The PER.C6®® cells were prepared according to the procedure outlined in WO20088006494. The Xₜ was 98.8×10⁶ cells/ml on day 13 of the reactor. The material was diluted to 75×10⁶ cells/ml with Dulbecco's PBS (5 mS/cm) to a final volume of 20 ml. The anion exchange material was added as a 50/50 slurry (total volume was 24 ml) and the cells were allowed to settle until the pellet volume was constant (60 min).

Aliquots of the harvest and supernatant were analyzed by analytical protein A chromatography to determine the product recovery. The recovery was determined by:

$$Recovery = \frac{Mass of MAb in Supernatant}{Mass of MAb in Harvest} \qquad ( 2 )$$

The analyses were corrected for the biomass when necessary, i.e. when the cell density is extremely high, the cells contribute significantly to the working volume.

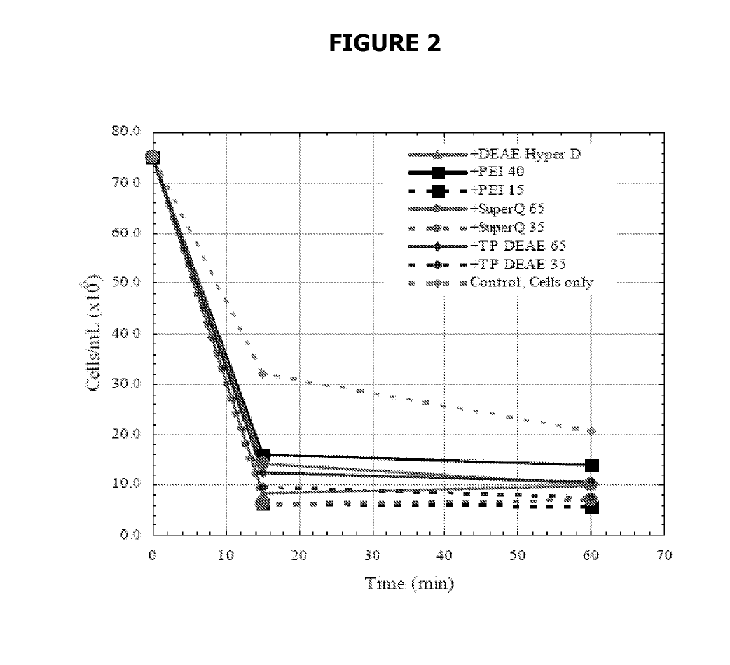

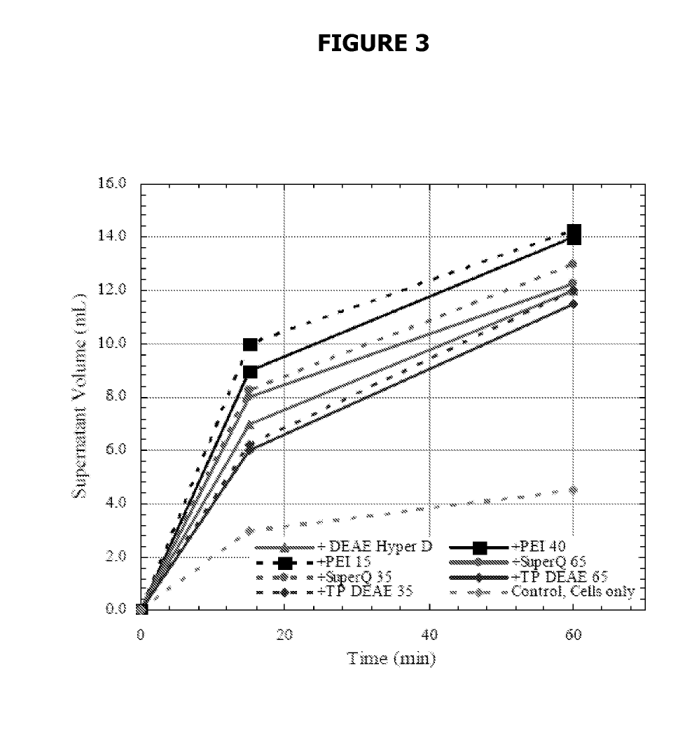

Experiments using CM Hyper D, a cation exchange chromatographic material, showed no advantage over the control. In the case of Hyper Z, the particle density was too great to facilitate settling. The resin immediately settled to the bottom of the vessel and no enhanced cell settling was observed. FIGS. 2 and 3 present the data for the anion exchange materials with positive results.

FIG. 2 shows the supernatant cell density as a function of time for each anion exchange material as well as the control where no anion exchange material was added. Accelerated cell settling was observed in each case compared with the control. The smaller particle size appears to decrease the supernatant cell density below 10×10⁶ cells/ml, where as the larger particle size decreases the cell density to 11−15×10⁶ cells/ml.

FIG. 3 shows the supernatant volume versus time for each anion exchange material. Addition of the Si-PEI material results in the largest amount of supernatant volume, which corresponds to the most compact pellet. The pellet accounted for 40% of the total volume in this case, whereas the pellet accounted for 63% of the total volume in the control. The Si-PEI materials have a greater density than the methacrylate and agarose based materials, which apparently allows for more compact pellets and faster settling rates. The ceramic Hyper D materials have an intermediate density with corresponding intermediate settling rates and pellet volumes.

Aliquots of the harvest and supernatant were analyzed by HCP ELISA to determine the reduction of impurities. HCP ELISA is an immunological method consisting of polyclonal antibodies raised against host cell proteins, in this case PER.C6®® derived proteins. The polyclonal antibodies are then used to coat micro titer plates, which are then incubated with in-process samples. Following incubation the micro titer plates are incubated again with the same polyclonal antibodies, this time the antibodies are conjugated with an enzyme such as horseradish peroxidase. This two-step process results in the creation of a “sandwich” complex. The complex is then reacted with a chromogenic substrate and the HCP population is quantified based on the color intensity.

The reduction of HCPs was determined by:

$$Reduction of HCP = \frac{( μ g of HCP / mg of MAb ) in Supernatant}{( μ g of HCP / mg of MAb ) in Harvest} \qquad ( 3 )$$

The analyses were corrected for the volume of the biomass when necessary.

The product recovery and HCP reduction are summarized in Table 2. The addition of the resin increases the product recovery and significantly reduces the HCP levels in the semi-clarified media. The recovery and HCP reduction are determined by equations (2) and (3), respectively.

TABLE 2
Product recovery and HCP reduction for various anion exchangers.

| | Particle size | | HCP Reduction |
|---|---|---|---|
| Anion exchange material | (μm) | Recovery (%) | (%) |
| N/A⁽¹⁾ | — | 63.1 | 0 |
| Si-PEI | 40 | 74.4 | 20.1 |
| | 15 | 96.2 | 39.0 |
| Super Q | 65 | 80.0 | 34.0 |
| | 35 | 87.6 | 37.8 |
| TP DEAE | 65 | 83.6 | 26.8 |
| | 35 | 77.8 | 32.6 |
| DEAE Hyper D | 50 | 76.2 | 35.3 |

⁽¹⁾In a control experiment no anion exchange material is added.

### Example 6

### Product Recovery at Larger Scale

Scale up experiments were performed with cell culture material from reactor volumes of 1.7 L and 1.8 L, respectively. The reactors were diluted 1:1 with 7 mS/cm PBS and 10% (vol) Si-PEI was added to the diluted harvest. After 1-1.3 hours of settling, the cell density in the resulting supernatant was substantially lower in each case. The supernatant was decanted and the pellet was washed twice with 7 mS/cm PBS with 0.75 hour of settling after each wash (total process time <2.8 hours). Table 3 summarizes the results from this scale up work.

TABLE 3
Product recovery, HCP reduction (measured by ELISA assay), and DNA
reduction (measured by real time PCR) from Si-PEI treated cell culture broths.

| | | | Vol | Vol | | | |
|---|---|---|---|---|---|---|---|
| | | | media | media | | HCP | DNA |
| | Xₜ initial | Xₜ final | initial | final | Recovery | reduction | reduction |
| Reactor | (×10⁶ cells/ml) | (×10⁶ cells/ml) | (L) | (L) | (%) | (%) | (%) |
| 1 | 143 | 2.4 | 1.7 | 5.9 | 93 | 53 | 65 |
| 2 | 175 | 4.1 | 1.6 | 6.6 | 95 | 66 | 59 |

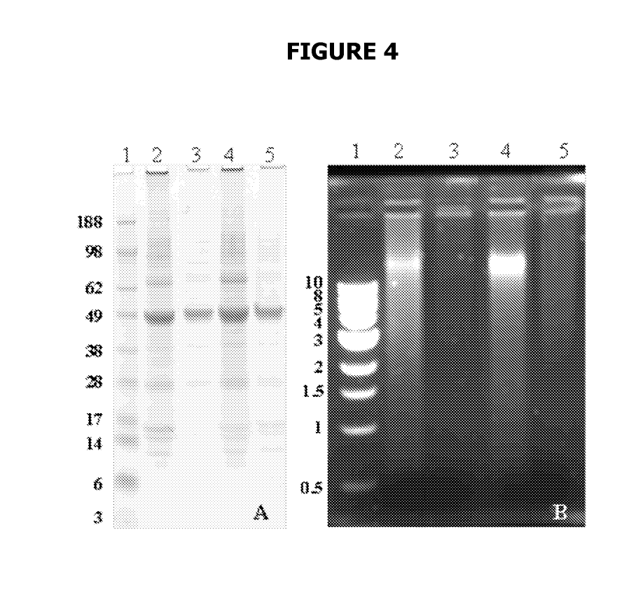

FIG. 4 shows the SDS-PAGE and agarose gels of samples taken from each of the two reactors. As evidenced by the gels, the levels of HCP and DNA in the Si-PEI treated samples are much lower than the levels initially in the harvest. Equivalent volumes were loaded into each lane for both gels. Additionally, the samples were pre-treated with protein A to remove the monoclonal antibody.

Table 4 below presents the % viability of the cell broths use din Examples 1-6.

TABLE 4

| | Xₜ | Xᵥ (viable | |
|---|---|---|---|
| | (total cells × | cells × | |
| Example | 10⁶/mL) | 10⁶/mL) | % Viability |
| 1 | 4.3 | 4.2 | 98 |
| 2 | 63.5 | 59.6 | 94 |
| 3 | 123.0 | 115.6 | 94 |
| 4-1* | 75.0 | 73.0 | 97 |
| 4-2* | 120.0 | 112.9 | 94 |
| 5 | 98.8 | 95.5 | 94 |
| 6-1* | 143.0 | 122.0 | 85 |
| 6-2* | 175.0 | 136.0 | 78 |

*Examples 4 and 6 describe material from two bioreactors.

### Example 7

### Purification of Desired Biological Substance

A cell culture harvest with initial cell density of 175×10⁶ cells/mL was diluted to ˜75×10⁶ cells/mL with PBS (Initial volume of 1.7 L). Following dilution Si-PEI chromatography media were added to the harvest (0.1 L of Si-PEI resin per L of diluted harvest). The cells were allowed to settle for ˜60 minutes. The product containing supernatant was decanted and the settled cells were washed twice with PBS. The initial supernatant was pooled together with the two washes to maximize product recovery (˜95%). The combined pool contains less than 5×10⁶ cells/ml, and the HCP content is reduced by 59% (see Reactor 2 in Table 3 above).

The product recovered after the Si-PEI settling is further purified by depth filtration. Depth filtration consist of a primary filter (typically 10/5 μm pore size) used for further reduction of the cell mass, followed by a secondary filter (typically 3/1 μm pore size) that removes smaller particles and prepares the clarified harvest for sterile filtration typically through a gradient 0.8/0.2 μm filter. The depth filtration train can be Millipore Millistak+HC filters containing media such as D0HC (primary) followed by X0HC (secondary) or CUNO ZetaPlus filters containing media such as 10M02 (primary) followed by 60ZA05A (secondary). The hydraulic capacity of the depth filter is greatly increased when the cell harvest media has been treated with Si-PEI. In either case the clarified harvest is further filtered through 0.8/0.2 μm filters (Supor, Pall).

In addition an 85% HCP reduction was observed through the secondary filter during depth filtration. Reduction in HCP through the secondary filter could be attributed to the charged nature of these filters and has been previously reported in the literature (Yigzaw Y, Piper R, Tran M, Shukla AA. 2006. Exploitation of the Adsorptive Properties of Depth Filters for Host Cell Protein Removal during Monoclonal Antibody Purification. Biotechnology Progress 22(1):288-296.).

The clarified material is further purified by Cation Exchange Chromatography such as GigaCap S (Tosoh). The monoclonal antibody (product) is immobilized on the resin at a capacity of >95 g/L of chromatography media. The conditions used for immobilizing the antibody are slightly acidic (pH˜5.3) and conductivity of ˜4.5 mS/cm. After binding, the antibody is washed with equilibration buffer and finally eluted with a buffer step containing 100 mM sodium chloride. An additional reduction in HCP content (78%) is obtained by this step.

The eluted antibody can be further purified by a combination of chromatography and filtration techniques until the required purity specifications are met. The overall reduction in Host Cell Proteins through the CEX step is summarized in Table 5 below:

TABLE 5

| | HCP | % |
|---|---|---|
| | (μg/mg MAb) | HCP Clearance |
| Cell Culture Harvest | 200 | 0 |
| Post-PEI Cell Settling | 81 | 59 |
| Post Depth Filtration | 12 | 85 |
| Post CEX Capture | 2.8 | 78 |
| Overall | | 99 |

1. A method for the clarification of a cell broth comprising the steps of:
(a) forming a mixture by contacting a particulate anion exchange material with a cell broth consisting essentially of culture medium, a desired biological substance having an overall positive charge in said cell broth, host cells that have produced said desired biological substance and being substantially viable, and host cell proteins,
(b) incubating said mixture for an adequate time to result in the formation of a cell pellet containing substantially all of said host cells and said particulate anion exchange material, and a supernatant layer containing said desired biological substance, and
(c) separating the resulting cell pellet from said supernatant layer.

2. A method according to claim 1 wherein less than about 22% of said host cells are dead.

3. A method according to claim 2 wherein less than about 10% of said host cells in said cell broth are dead.

4. A method according to claim 3 wherein less than about 5% of said host cells in said cell broth are dead.

5. A method according to claim 1 wherein said particulate anion exchange material has a specific particle density of between about 1.4 and about 3 g/ml.

6. A method according to claim 1 wherein said cells in said cell broth are at an initial cell density of about 15×10⁶ cell/ml to about 130×10⁶ cell/ml.

7. A method according to claim 6 wherein prior to step (a), said host cells in said cell broth are diluted to a cell density of about 15×10⁶ cell/ml to about 80×10⁶ cells/ml.

8. A method according to claim 7 wherein the density of cells remaining in said supernatant layer is reduced by at least about 87 percent to from about 2×10⁶ cell/ml to about 15×10⁶ cell/ml.

9. A method for the recovery of a desired biological substance from a cell broth containing host cell secreting said desired biological substance, comprising the steps of:
(a) forming a mixture by contacting a particulate anion exchange material with a cell broth containing cell culture medium, host cell proteins, substantially viable mammalian host cells, and a desired biological substance secreted by said mammalian host cells, said substance having an overall positive charge in said cell broth, wherein said mammalian host cells are present at an initial cell density of at least about 15×10⁶ cell/ml,
(b) incubating said mixture for an adequate time to result in the formation of a cell pellet and a supernatant layer containing the desired biological substance and having a reduced cell density and host cell protein content,
(c) separating the resulting cell pellet from the supernatant layer, and
(d) extracting the desired biological substances from the supernatant layer.

10. Method according to claim 9, wherein the resulting cell pellet is further processed by the following steps:
(e) re-suspending said resulting cell pellet from step (c) after separation of said supernatant layer in an aqueous salt solution to form a second mixture,
(f) incubating said second mixture for an adequate time to allow the formation of a second cell pellet and a second supernatant layer from said re-suspended cell pellet,
(g) separating said second cell pellet from said second supernatant layer, and
(h) extracting the desired biological substances from said second supernatant layer.

11. Method according to claim 10, wherein step (e) through (h) are repeated.

12. Method according to claim 10, wherein the aqueous salt solution is isotonic salt solution.

13. Method according to claim 10 wherein each of said supernatant layers are combined prior to extracting said desired biological substance.

14. Method according to claim 13 wherein said combined supernatant layer is subjected to depth filtration to further reduce host cell density and host cell protein content.

15. Method according to claim 14 wherein said desired biological substance is extracted from said filtered supernatant layers using cation exchange chromatography.

16. Method according to claim 1, wherein the desired biological substance is an immunoglobulin or parts thereof.

17. Method according to claim 1, wherein said contacting is with a cell broth wherein the total cell density is no less than 90% of the maximum total cell density.
